(19) United States
(12) Patent Application Publication
(10) Pub. No.: US 2020/0229993 A1
(43) Pub. Date: Jul. 23, 2020

(54) Biohazard Storage Assembly

(71) Applicants: Evon Bryant, Poland, OH (US); Valerie Bryant, Poland, OH (US)

(72) Inventors: Evon Bryant, Poland, OH (US); Valerie Bryant, Poland, OH (US)

(21) Appl. No.: 16/250,459

(22) Filed: Jan. 17, 2019

(51) Int. Cl. A61F 13/551; B65F 1/02; B65F 1/16

(52) U.S. Cl. CPC A61F 13/5515; A61F 13/5518; B65F 2240/164; B65F 1/16; B65F 1/02

(57) ABSTRACT

A biohazard storage assembly for transporting soiled feminine hygiene products prior to disposal includes a pad is comprised of a fluid absorbent material thereby facilitating the pad to be employed for feminine hygiene. A plurality of canisters is provided and each of the canisters is comprised of a fluid impermeable material. The pad is positionable in a respective one of the canisters when the pad has absorbed bodily fluids. In this way the respective canister inhibits the bodily fluids from contacting a person. A plurality of caps is provided and each of the caps is positionable on a respective one of the canisters for closing the respective canister.

### CROSS-REFERENCE TO RELATED APPLICATIONS

### Statement Regarding Federally Sponsored Research or Development

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to biohazard devices and more particularly pertains to a new biohazard device for transporting soiled feminine hygiene products prior to disposal.

### BRIEF SUMMARY OF THE INVENTION

An embodiment of the disclosure meets the needs presented above by generally comprising a pad is comprised of a fluid absorbent material thereby facilitating the pad to be employed for feminine hygiene. A plurality of canisters is provided and each of the canisters is comprised of a fluid impermeable material. The pad is positionable in a respective one of the canisters when the pad has absorbed bodily fluids. In this way the respective canister inhibits the bodily fluids from contacting a person. A plurality of caps is provided and each of the caps is positionable on a respective one of the canisters for closing the respective canister.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a plurality of canisters and caps of a biohazard storage assembly according to an embodiment of the disclosure.

FIG. 2 is a top perspective view of an embodiment of the disclosure.

FIG. 3 is a front cut-away view of an embodiment of the disclosure.

### DETAILED DESCRIPTION OF THE INVENTION

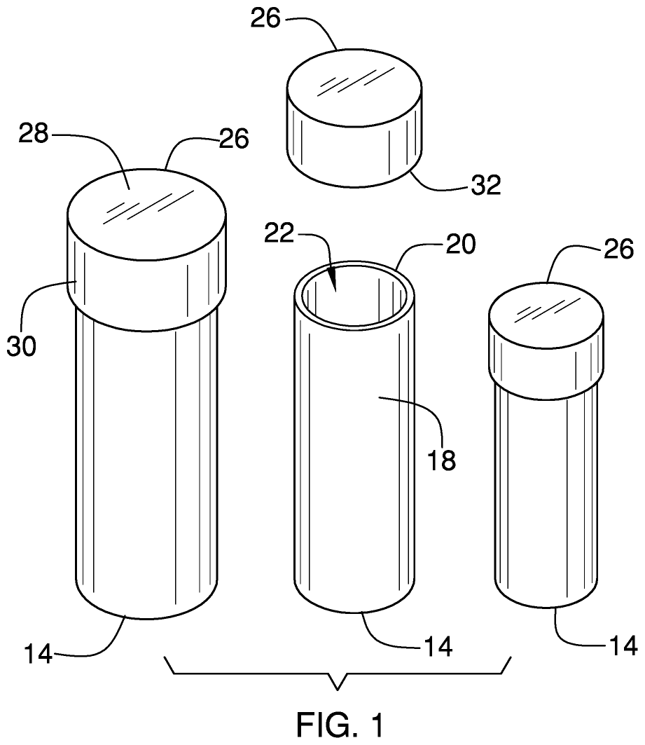

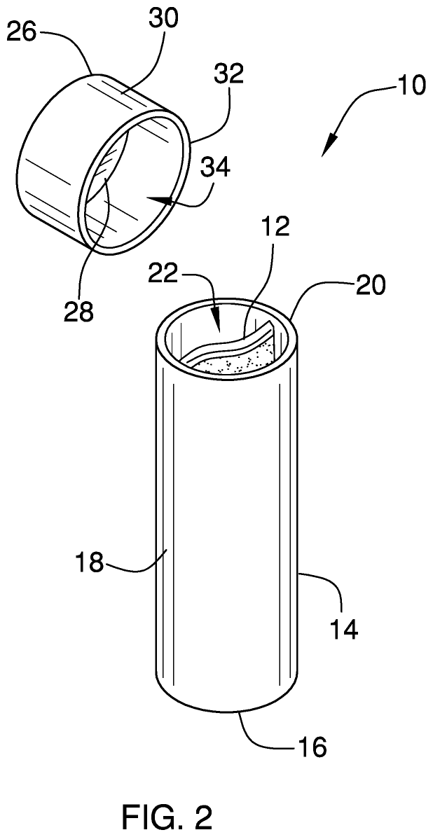

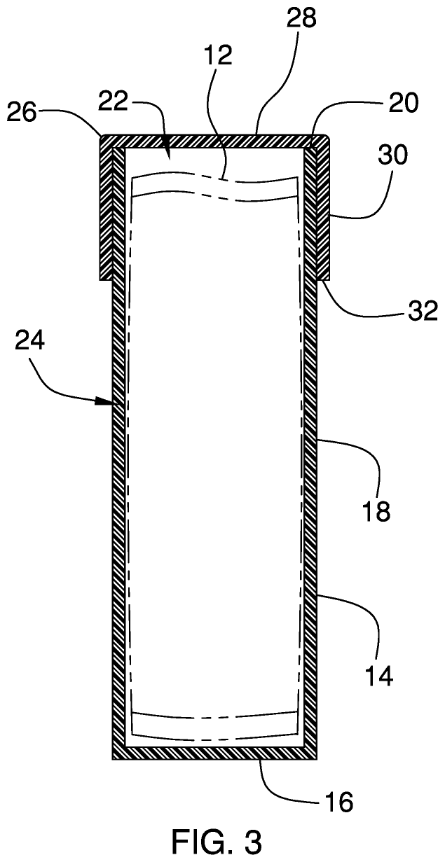

With reference now to the drawings, and in particular to FIGS. 1 through 3 thereof, a new biohazard device embodying the principles and concepts of an embodiment of the disclosure and generally designated by the reference numeral 10 will be described.

As best illustrated in FIGS. 1 through 3, the biohazard storage assembly 10 generally comprises a pad 12 that is comprised of a fluid absorbent material thereby facilitating the pad 12 to be employed for feminine hygiene. The pad 12 may be a tampon, a maxi pad or any other type of feminine hygiene product. A plurality of canisters 14 is provided and each of the canisters 14 is comprised of a fluid impermeable material. The pad 12 is positionable in a respective one of the canisters 14 when the pad 12 has absorbed bodily fluids. In this way the respective canister 14 inhibits the bodily fluids from contacting a person. Additionally, the pad 12 in the canister 14 can be safely and discretely transported until the pad 12 can be permanently discarded.

Each of the canisters 14 has a unique size with respect to each other for accommodating a variety of sizes of pads 12. Each of the canisters 14 has a bottom wall 16 and an outer wall 18 extending upwardly therefrom, and the outer wall 18 has a distal edge 20 with respect to the bottom wall 16 defining an opening 22 into the canisters 14. Each of the canisters 14 is infused with and thusly releases a chemical scent 24. Thus, the chemical scent 24 masks odors associated with the bodily fluids from the pad 12 when the pad 12 is positioned in the respective canister 14. The chemical scent 24 may be a floral scent, an antiseptic scent or any other scent that can thoroughly mask odors associated with bodily fluids.

A plurality of caps 26 is included and each of the caps 26 is positionable on a respective one of the canisters 14 for closing the respective canister 14. Each of the caps 26 has an upper wall 28 and a perimeter wall 30 extending downwardly therefrom, and the perimeter wall 30 of each of the caps 26 is continuously arcuate such that each of the caps 26 has a cylindrical shape. The perimeter wall 30 of each of the caps 26 has a lower edge 32 defining an opening 34 into the caps 26 and the perimeter wall 30 has an inside surface 36. The opening 22 in each of the caps 26 insertably receives the distal edge 20 of the respective canister 14 and the inside surface 36 of the perimeter wall 30 engages the outer wall 18 of the respective canister 14. Additionally, the distal edge 20 of outer wall 18 of the respective canister 14 abuts the upper wall 28 of the cap 26 when the cap 26 is seated on the respective canister 14.

In use, the pad 12 is placed into the respective canister 14 when the pad 12 has become soaked with the bodily fluids. A respective cap 26 is placed on the respective canister 14 for closing the respective canister 14. Thus, the pad 12 may be discretely and safely transported, in a purse or the like, until such time that the pad 12 can be permanently discarded. Additionally, the chemical scent 24 in the respective canister 14 masks any potential odors associated with the bodily fluids. The respective canister 14, respective cap 26 and the pad 12 inside the respective canister 14 are permanently discarded together.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A biohazard storage assembly being configured to store material soaked with bodily fluid for disposal, said assembly comprising:
a pad being comprised of a fluid absorbent material wherein said pad is configured to be employed for feminine hygiene;
a plurality of canisters, each of said canisters being comprised of a fluid impermeable material, said pad being positionable in a respective one of said canisters when said pad has absorbed bodily fluids wherein said respective canister is configured to inhibit the bodily fluids from contacting a person; and
a plurality of caps, each of said caps being positionable on a respective one of said canisters for closing said respective canister.

2. The assembly according to claim 1, wherein:
each of said canisters having a unique size with respect to each other for accommodating a variety of sizes of pads, each of said canisters having a bottom wall and an outer wall extending upwardly therefrom, said outer wall having a distal edge with respect to said bottom wall defining an opening into said canisters; and
each of said canisters being infused with and thusly releasing a chemical scent thereby facilitating said chemical scent to mask odors associated with the bodily fluids from said pad when said pad is positioned in said respective canister.

3. A biohazard storage assembly being configured to store material soaked with bodily fluid for disposal, said assembly comprising:
a pad being comprised of a fluid absorbent material wherein said pad is configured to be employed for feminine hygiene;
a plurality of canisters, each of said canisters being comprised of a fluid impermeable material, said pad being positionable in a respective one of said canisters when said pad has absorbed bodily fluids wherein said respective canister is configured to inhibit the bodily fluids from contacting a person, each of said canisters having a unique size with respect to each other for accommodating a variety of sizes of pads, each of said canisters having a bottom wall and an outer wall extending upwardly therefrom, said outer wall having a distal edge with respect to said bottom wall defining an opening into said canisters, each of said canisters being infused with and thusly releasing a chemical scent thereby facilitating said chemical scent to mask odors associated with the bodily fluids from said pad when said pad is positioned in said respective canister; and
a plurality of caps, each of said caps being positionable on a respective one of said canisters for closing said respective canister.

4. The assembly according to claim 3, wherein each of said caps has an upper wall and a perimeter wall extending downwardly therefrom, said perimeter wall of each of said caps being continuously arcuate such that each of said caps has a cylindrical shape, said perimeter wall of each of said caps having a lower edge defining an opening into said caps and an inside surface.

5. The assembly according to claim 4, wherein said opening in each of said caps insertably receives said distal edge of said respective canister having said inside surface of said perimeter wall engaging said outer wall of said respective canister, said distal edge of outer wall of said respective canister abutting said upper wall of said cap when said cap is seated on said respective canister.

6. A biohazard storage assembly being configured to store material soaked with bodily fluid for disposal, said assembly comprising:
a pad being comprised of a fluid absorbent material wherein said pad is configured to be employed for feminine hygiene;
a plurality of canisters, each of said canisters being comprised of a fluid impermeable material, said pad being positionable in a respective one of said canisters when said pad has absorbed bodily fluids wherein said respective canister is configured to inhibit the bodily fluids from contacting a person, each of said canisters having a unique size with respect to each other for accommodating a variety of sizes of pads, each of said canisters having a bottom wall and an outer wall extending upwardly therefrom, said outer wall having a distal edge with respect to said bottom wall defining an opening into said canisters, each of said canisters being infused with and thusly releasing a chemical scent thereby facilitating said chemical scent to mask odors associated with the bodily fluids from said pad when said pad is positioned in said respective canister; and
a plurality of caps, each of said caps being positionable on a respective one of said canisters for closing said respective canister, each of said caps having an upper wall and a perimeter wall extending downwardly therefrom, said perimeter wall of each of said caps being continuously arcuate such that each of said caps has a cylindrical shape, said perimeter wall of each of said caps having a lower edge defining an opening into said caps and an inside surface, said opening in each of said caps insertably receiving said distal edge of said respective canister having said inside surface of said perimeter wall engaging said outer wall of said respective canister, said distal edge of outer wall of said respective canister abutting said upper wall of said cap when said cap is seated on said respective canister.
